(19) United States
(12) Patent Application Publication
(10) Pub. No.: US 2010/0152709 A1
(43) Pub. Date: Jun. 17, 2010

(54) Dysfunction Resistant Catheter Systems and Associated Methods

(75) Inventors: Thomas J. Maginot, Crown Point, IN (US); Paul J. Maginot, Fishers, IN (US)

Correspondence Address: MAGINOT, MOORE & BECK, LLP;CHASE TOWER, 111 MONUMENT CIRCLE, SUITE 3250, INDIANAPOLIS, IN, 46204, US

(73) Assignee: CathLogic, Inc., Crown Point, IN (US)

(21) Appl. No.: 12/638,974

(22) Filed: Dec. 15, 2009

Related U.S. Application Data
(60) Provisional application No. 61/122,423, filed on Dec. 15, 2008.

(51) Int. Cl. A61M 25/00

(52) U.S. Cl. 604/523

(57) ABSTRACT

A catheter assembly includes a first line having (i) a first proximal end portion defining a first opening, (ii) a first distal end portion defining a second opening, and (iii) a first fluid passage extending between the first opening and the second opening. The catheter assembly includes a second line having (i) a second proximal end portion defining a third opening, (ii) a second distal end portion defining a fourth opening, and (iii) a second fluid passage extending between the third opening and the fourth opening. The catheter assembly further includes an actuator movable between a first location and a second location. In addition, the catheter assembly includes a linkage connected between the actuator and the first distal end portion of the first line. The first distal end portion of the first line is movable in relation to the second line between (i) a first position in which the first distal end portion is positioned in abutment with the second line, and (ii) a second position in which the distal end portion is spaced apart from the second line. Movement of the actuator from the first location to the second location causes movement of the first distal end portion from the first position to the second position.

This application claims the benefit of U.S. Provisional Application Ser. No. 61/122,423, filed Dec. 15, 2008, the disclosure of which is herein totally incorporated by reference in its entirety

### CROSS REFERENCE

Cross reference is made to the following U.S. Pat. Nos. 5,989,213; 6,156,016; 6,190,371; 6,475,207; 6,585,705; 6,723,084; 6,743,218; 7,008,412, the disclosures of each of the above-identified patents is herein totally incorporated by reference in their entirety. Cross reference is further made to U.S. patent application Ser. No. 10/005,277 (Patent Application Publication No. US 2002/0091362 A1); Ser. No. 10/857,185 (U.S. Patent Application Publication No. 2005/0096609 A1); Ser. No. 10/857,621 (U.S. Patent Application Publication No 2005/0059925 A1); Ser. No. 11/375,856 (U.S. Patent Application Publication No. US 2006/0161118 A1); and Ser. No. 11/375,881 (U.S. Patent Application Publication No. US 2006/0161119 A1), the disclosures of each of the above-identified published patent applications is herein totally incorporated by reference in their entirety.

### BACKGROUND

The present disclosure relates generally to catheter systems, and more particularly to catheter systems that may be used in the performance of a medical procedure such as a dialysis procedure. A problem being address by the present invention is catheter dysfunction due to fibrin sheath formation. This problem is well known in the art and can be understood from one or more of the patents and published patent applications identified above.

### SUMMARY

In accordance with one embodiment of the disclosure, there is provided a catheter assembly that includes a first line having (i) a first proximal end portion defining a first opening, (ii) a first distal end portion defining a second opening, and (iii) a first fluid passage extending between the first opening and the second opening. The catheter assembly includes a second line having (i) a second proximal end portion defining a third opening, (ii) a second distal end portion defining a fourth opening, and (iii) a second fluid passage extending between the third opening and the fourth opening. The catheter assembly further includes an actuator movable between a first location and a second location. In addition, the catheter assembly includes a linkage connected between the actuator and the first distal end portion of the first line. The first distal end portion of the first line is movable in relation to the second line between (i) a first position in which the first distal end portion is positioned in abutment with the second line, and (ii) a second position in which the distal end portion is spaced apart from the second line. Movement of the actuator from the first location to the second location causes movement of the first distal end portion from the first position to the second position.

### BRIEF DESCRIPTION OF THE DRAWINGS

FIG. 1 is a front elevational view of a catheter assembly of the present disclosure;

FIG. 2 is a top elevational view of the catheter assembly of FIG. 1;

FIGS. 3-5 are front elevational views of the catheter assembly of FIG. 1 showing with its distal portion being actuated to an operative state;

FIG. 6 is a cross-sectional view taken along lines 6-6 of FIG. 3;

FIG. 6A is a side elevational view of the first distal end portion of FIG. 3;

FIG. 7 is a cross-sectional view taken along lines 7-7 of FIG. 3;

FIG. 8 is a cross-sectional view taken along lines 8-8 of FIG. 3;

FIG. 9 shows a cross-sectional view of a first alternative embodiment of a catheter system;

FIG. 10 shows a cross-sectional view of a another alternative embodiment of a catheter system;

FIGS. 11-15 show various views of still another embodiment of a catheter system; and

FIG. 16 shows a distal portion of yet still another alternative embodiment of a catheter system.

### DESCRIPTION OF THE PREFERRED EMBODIMENTS

While the catheter system of the present disclosure is susceptible to various modifications and alternative forms, specific embodiments thereof have been shown by way of example in the drawings and will herein be described in detail. It should be understood, however, that there is no intent to limit the catheter system to the particular forms disclosed, but on the contrary, the intention is to cover all modifications, equivalents, and alternatives falling within the spirit and scope of the invention.

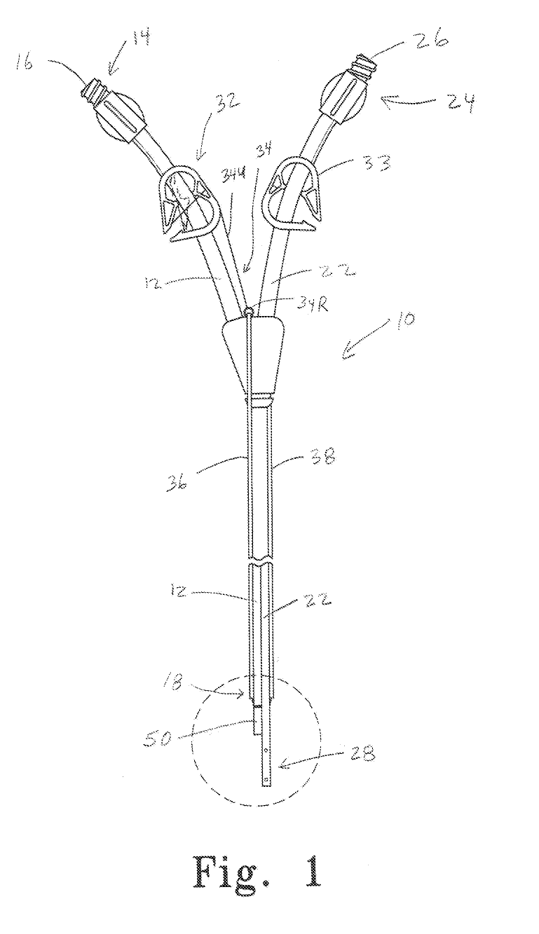

Turning now to FIGS. 1-8, there is shown a catheter assembly 10. The catheter system 10 includes a first arterial line 12 having (i) a first proximal end portion 14 defining a first opening 16, (ii) a first distal end portion 18 defining a second opening 20, and (iii) a first fluid passage 21 extending between the first opening 16 and the second opening 20. The catheter system further includes a second venous line 22 having (i) a second proximal end portion 24 defining a third opening 26, (ii) a second distal end portion 28 defining a fourth opening 30, and (iii) a second fluid passage 31 extending between the third opening 26 and the fourth opening 30.

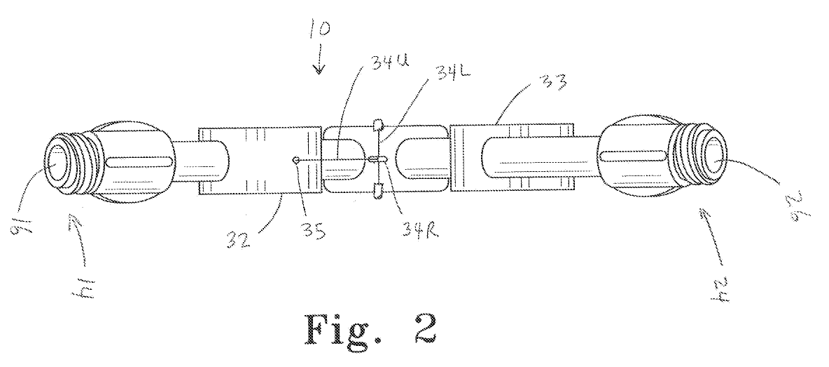

The catheter system 10 further includes an actuator 32 movable between a first location (shown in phantom in FIG. 1) and a second location (shown in solid in FIG. 1). In addition, the catheter system 10 further includes a linkage 34 connected between the actuator 32 and the first distal end portion 18 of the first line 12. The linkage 34 includes an upper linkage 34U, a ring 34R, and a lower linkage 34L. The catheter system 10 further includes a first guide tube 36 bonded to the first line 12 and a second guide tube 38 bonded to the second line 22. The lower linkage 34L (see FIG. 2) is located in the first guide tube 36 and the second guide tube 38. However, as shown in FIG. 2, a segment of the lower linkage 34L is exposed, and the ring 34R is positioned around the exposed linkage segment. The first guide tube 36 and the second guide tube 38 possess a French size of 3 or less, and preferably 2. The upper linkage 34U is preferably nylon (polyamide) suture line. The lower linkage 34L is also preferably nylon (polyamide) suture line.

In order to facilitate sliding of the lower linkage 34L in the tubes 36, 38, each of the tubes 36, 38 may contain a hollow outer cable or wire. The lower linkage 34L may slide within the hollow outer cable or wire. This type of mechanical arrangement is known in the mechanical arts as a Bowden cable in which mechanical force or energy is transmitted by the movement of the inner cable relative to the hollow outer cable. The hollow outer cable or wire may be made of metal such as titanium (or other biocompatible metals) or a plastic material such as polyethylene or polyurethane (or other biocompatible plastics). Thus, the lower linkage 34L and the hollow outer cable or wire may be configured to form a Bowden cable similar to the Bowden cable described in U.S. Patent Application Publication Nos. 2005/0096609 A1 and 2005/0059925 A1.

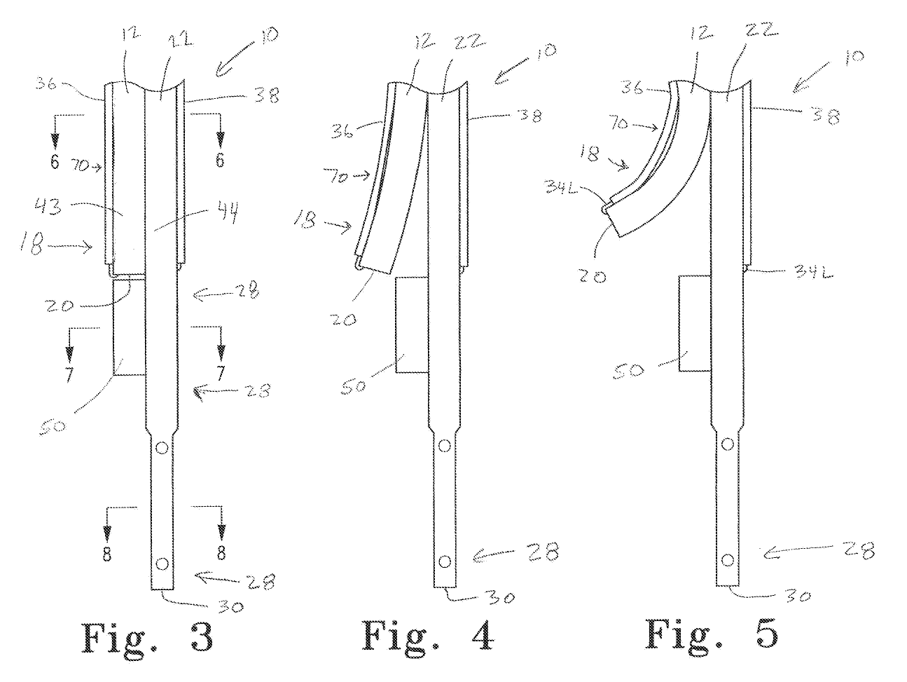

The first distal end portion 18 of the first line 12 is movable in relation to the second line 22 between (i) a first position in which the first distal end portion 18 is positioned in abutment with the second line 22 as shown in FIG. 3, and (ii) a second position in which the distal end portion 18 is spaced apart from the second line 22 as shown in FIG. 5. FIG. 4 shows the first distal end portion 18 positioned at an intermediate position as the first distal end portion 18 moves from its first position (FIG. 3) to its second position (see FIG. 5). Movement of the actuator 32 from its first location (shown in phantom in FIG. 1) to its second location (shown in solid in FIG. 1) causes movement of the first distal end portion from the first position (shown in FIG. 3) to its second position (shown in FIG. 5).

The actuator 32 includes a clamp 32 positioned around the first line 12, while another clamp 33 is positioned around the second line 22. The clamp 32 is movable between a clamped configuration (shown in phantom in FIG. 1) and an unclamped configuration (shown in solid in FIG. 1). Movement of the clamp 32 from the clamped configuration to the unclamped configuration causes movement of the distal end portion 18 from the first position (shown in FIG. 3) to the second position (shown in FIG. 5). In other words, as the clamp 32 opens, the distal end portion 18 of the arterial line 12 advances away from the venous line 22. Similarly, as the clamp 32 closes, the distal end portion 18 of the arterial line 12 advances towards the venous line 22.

Fluid flow through the arterial line 12 is prevented when the clamp 32 is in the clamped configuration, while fluid flow though the arterial line 12 is enabled when the clamp 32 is in the unclamped configuration. The clamp 33 functions with the venous line 22 in an analogous manner.

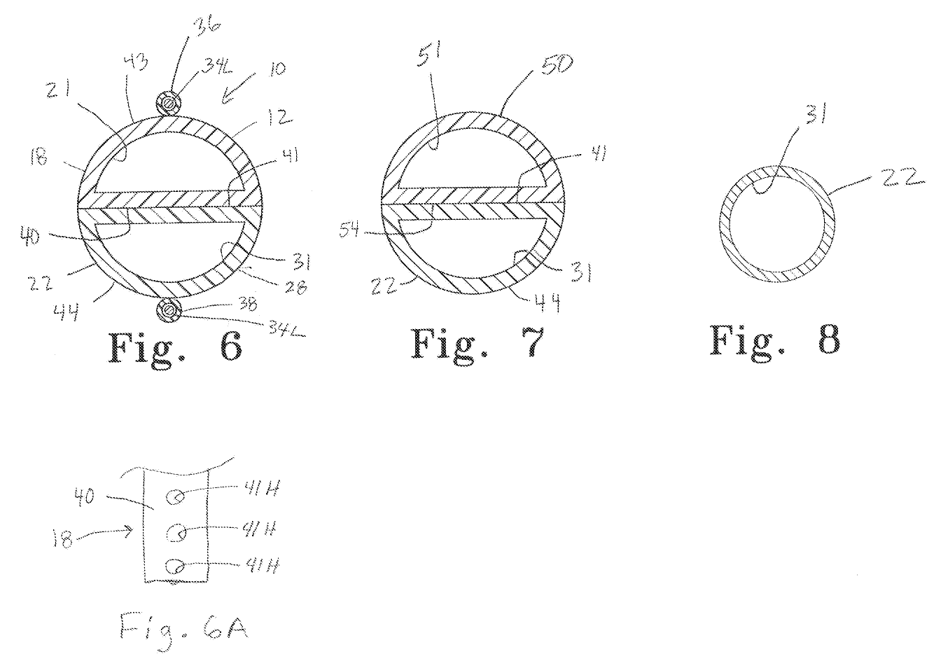

The first distal end portion 18 of the first line 12 is D-shaped as shown in FIG. 6, and defines a first flat wall 40. The second distal end portion 28 of the second line 22 is also D-shaped and defines a second flat wall 41. Note that this D-shaped section of the second distal end portion occurs a location that is spaced apart from the distal end of the second line 22. The first flat wall 40 is juxtaposed to the second flat 41 wall when the first distal end portion 18 of the first line 12 is positioned in the first position as shown in FIG. 3. The first flat wall 40 is spaced apart from the second flat wall 41 when the first distal end portion 18 of the first line 12 is positioned in the second position as shown in FIG. 5.

The first flat wall 40 has defined therein a first plurality of holes, and the second flat wall 41 has defined therein a second plurality of holes 41H as shown in FIG. 6A. The first distal end portion 18 of the first line 12 further defines a first curved wall 43 to which the first flat wall 40 is attached. The first curved wall 43 is free of holes. The second distal end portion 28 of the second line 22 further defines a second curved wall 44 to which the second flat wall 41 is attached. The second flat wall 41 is free of holes. The first flat wall 40 and the first curved wall 43 collectively define the second opening 20.

The linkage 34 includes a first segment of suture 34L having (i) a first end coupled to the first distal end portion 18 by sewing or bonding, (ii) a second end coupled to the second line 22 by sewing or bonding, and (iii) a central portion interposed between the first suture end and the second suture end. The linkage 34 also includes a second segment of suture 34U having (i) a first end coupled to the actuator 32 by tying a knot or bonding and (ii) a second end coupled to the central portion of the first segment of suture 34L via a ring 34R. Note that the suture 34U extends through a hole 35 defined in the actuator or clamp 32 as shown in FIG. 2 so that a middle segment of the suture 34U may be advanced through the hole 35 during clamping and unclamping of the clamp 32.

The linkage 34 further includes the ring 34R and defines a through-hole. The second end of the second segment of suture 34U is attached to the ring by tying a knot or bonding. The central portion of the first segment of suture 34L extends through the through-hole of the ring 34R to allow advancement of the suture 34L through the ring 34R.

The first guide tube 36 defines a first guide lumen, the first guide tube being secured to the first line 12, while the second guide tube 38 defines a second guide lumen, the second guide tube being secured to the second line 22. The first segment of suture 34L extends through both the first guide lumen 36 and the second guide lumen 38. Note that the distal ends of the guide tubes 36, 38 are plugged (e.g. by silicon) to prevent fluid such as blood from leaking or otherwise entering the distal ends of the guide tubes 36, 38.

The catheter system further includes a tube segment 50 attached to an outer wall of the second line 22. The second opening 20 of the first distal end portion 18 of the first line 12 is located proximal to the tube segment 50. The tube segment 50 defines a passageway 51 therethrough as shown in FIG. 7. The passageway 51 of the tube segment 50 is aligned with the first fluid passage 21 of the first line 12. The tube segment 50 may be positioned even closer to the distal end portion 18 than shown in FIGS. 3-5 so as to inhibit blood entering the distal opening 20 of the arterial line 12.

The tube segment 50 defines a flat wall 54. The outer wall of the second line 22 defines the second flat wall 41, and the flat wall 54 is secured to the second flat wall 41.

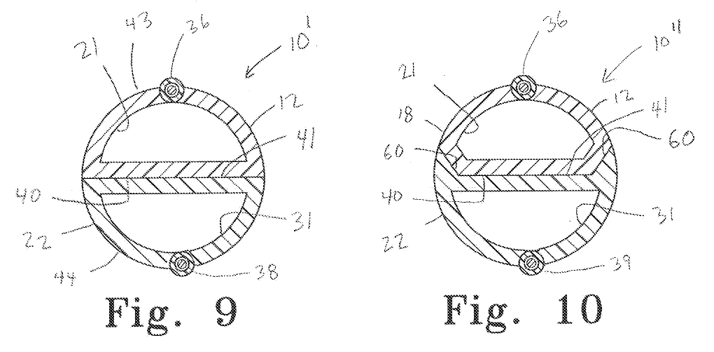

FIG. 9 shows a first alternative embodiment of a catheter system 10′ wherein the first guide tube 36 is imbedded in the wall of the first line 12 instead of being merely bonded to an outer surface thereof. Alternatively, the catheter system 10 may omit the first guide tube 36 and the second guide tube 38 altogether, and instead have a first guide channel formed in the first line 12 (in addition to the first passage 21) and a second guide channel formed in the second line 22 (in addition to the second passage 31). The lower linkage 34L would then be located in the first and second guide channels.

FIG. 10 shows another embodiment of a catheter system 10″ that is similar to the catheter system 10′ of FIG. 9, but showing the second line 22 defining a cradle 60 configured to receive a complementary configured distal end portion 18′ of the first line 12.

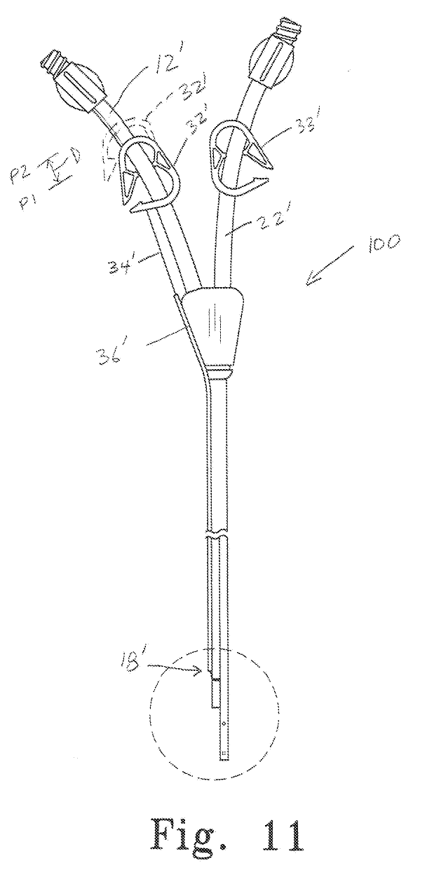

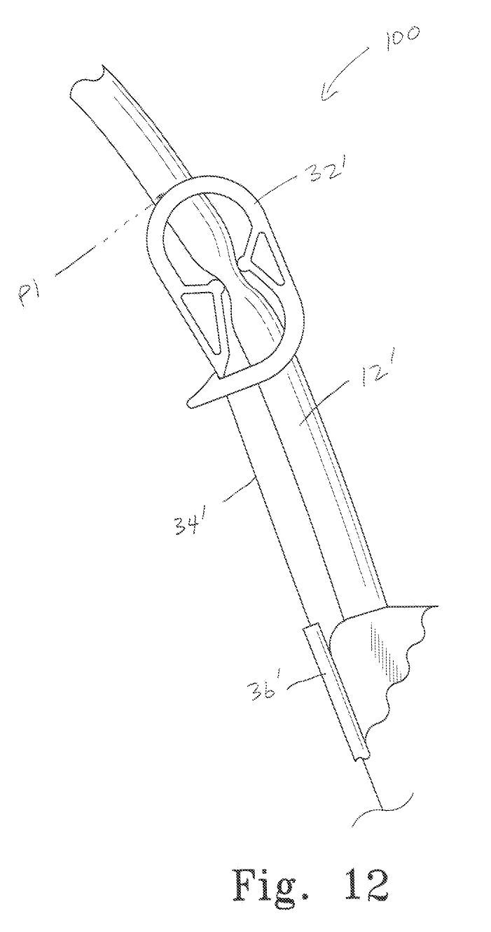

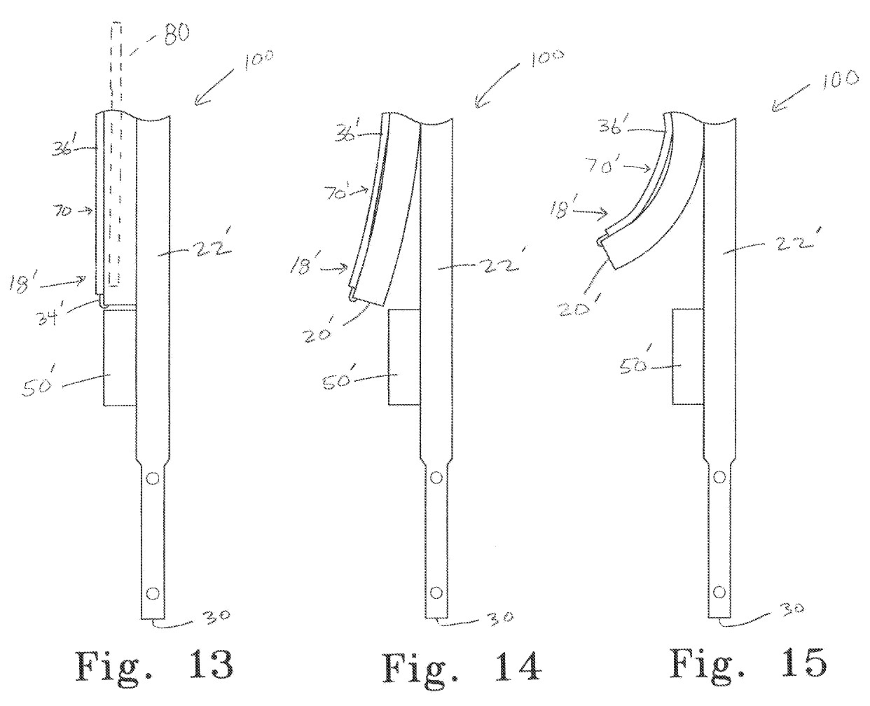

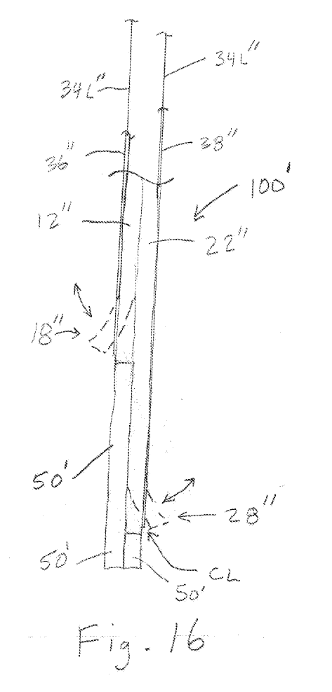

FIGS. 11-15 show yet another embodiment of a catheter system 100. The catheter system 100 is identical in construction and operation to the catheter system 10 of FIGS. 1-8 with some exceptions. For example, a first exception is the catheter system 100 includes only a single guide tube 36′ bonded to the first line 12′. Also, the linkage 34′ includes only a single suture segment connected between the distal end portion 18′ of the first line 12′ and the actuator 32′. In order to move the distal end portion 18′ of the first line 12′ from its position shown in FIG. 13 (similar to FIG. 3) to its position shown in FIG. 15 (similar to FIG. 5), a user would advance the actuator or clamp 32′ from its position P1 shown in solid in FIG. 11 to its position P2 shown in phantom in FIG. 11. Movement of the clamp 32′ from position P1 to position P2 displaces the clamp 32′ a distance of D. Advancement of the clamp 32′ in this manner occurs by sliding the clamp 32′ upwardly in relation to the arterial line 12. After being advanced to its position P2, the clamp 32′ remains located at position P2 by friction between the outer surface of the arterial line 12′ and the clamp 32′. Alternatively, the line 12′ may be provided with a detent (not shown) to assist in retention of the clamp 32′ at its position P2. Note that FIG. 12 shows the clamp 32′ clamped to the line 12′ at its first position P1.

Note that in the embodiments shown in FIGS. 1-8 and the embodiments shown in FIGS. 11-15, the catheter systems 10, 100 are constructed with the guide tubes 36, 36′ attached to the respective first lines 12, 12′ so that there exists an unbonded region 70, 70′ near the distal end portions 18, 18′. (See, e.g., FIG. 5.) This region is left unbonded in both embodiments so as to facilitate bending of the distal end portions 18, 18′ in relation to the second lines 22, 22′.

FIG. 16 shows yet another embodiment of a catheter system 100′. The catheter system 100′ is identical in construction and operation to the catheter system 10 of FIGS. 1-8 with some exceptions. For example, in the catheter system 100′ the connection location CL between the lower linkage 34L and the second line 22 is more distal so that it is very close to the distal end of the second line 22. Also, the tube 50 of the catheter system 10 is replaced with a double tube arrangement 50′. Then, movement of the actuator 32 from its first location (shown in phantom in FIG. 1) to its second location (shown in solid in FIG. 1) causes movement of the first distal end portions 18″, 28″ of the first and second lines 12″, 22″ from their respective first positions (shown in solid in FIG. 16) to their respective second positions (shown in phantom in FIG. 16).

In all the embodiments disclosed herein, an I-shaped spring 80 (see FIG. 13) may be secured to the distal end portions 18, 18′ of the first line 12, 12′ in order to assist the distal end portion 18, 18′ in returning to its pre-actuated state (e.g. shown in FIGS. 3 and 13).

It should be appreciated that all of the catheter systems disclosed herein (e.g. catheter systems 10, 10′, 10″, 100, 100′) are used in medical procedures in a very similar way to the use of the various retractable catheter systems disclosed in the above-identified patents and patent applications that were incorporated herein by reference (hereinafter “Incorporated Patent Documents”).

For example, a patient would have the catheter system 10 implanted in his or her body as disclosed in the Incorporated Patent Documents. Then, when a medical procedure such as a dialysis procedure is to be performed with the catheter system 10, the clamp 32 would be manipulated to move from its clamped state (shown in phantom in FIG. 1) to its unclamped state (shown in solid in FIG. 1). In response to this unclamping of the clamp 32, the distal end portion 18 moves from its deactuated state or position (shown in FIG. 3) to its actuated state or position (shown in FIG. 5). Once the distal end portion 18 is positioned as shown in FIG. 5 (and the catheter system 10 now properly coupled to a dialysis machine), a dialysis procedure or session is performed on the patient. After the dialysis procedure is complete (and the catheter system is properly flushed as is well known in the art), the clamp 32 is manipulated so that it moves from is unclamped configuration (shown in solid in FIG. 1) to its clamped configuration (shown in phantom in FIG. 1). In response to this clamping of the clamp 32, the distal end portion 18 moves from its actuated state or position (shown in FIG. 5) to its deactuated state or position (shown in FIG. 3).

As another example, with the catheter system 100 implanted in his or her body as discussed above, when a medical procedure such as a dialysis procedure is to be performed with the catheter system 100, the clamp 32′ would be manipulated to move from its clamped state (shown in FIG. 12) to its unclamped state (shown in solid in FIG. 11). Then, the clamp 32′ is slid upwardly over the arterial line 12′ from its position P1 (shown in solid in FIG. 11) to its position P2 (shown in phantom in FIG. 11). In response to this movement of the clamp 32′, the distal end portion 18′ moves from its deactuated state or position (shown in FIG. 13) to its actuated state or position (shown in FIG. 15). Once the distal end portion 18′ is positioned as shown in FIG. 5 (and the catheter system 100 now properly coupled to a dialysis machine), a dialysis procedure or session is performed on the patient. After the dialysis procedure is complete (and the catheter system is properly flushed as is well know in the art), the clamp 32′ is slid downwardly over the arterial line 12′ from its position P2 (shown in phantom in FIG. 11) to its position P1 (shown in solid in FIG. 11). In response to this movement of the clamp 32′, the distal end portion 18′ moves from its actuated state or position (shown in FIG. 15) to its deactuated state or position (shown in FIG. 13). When the clamp 32′ is at its position P1, the clamp 32′ is manipulated so that it moves from its unclamped configuration (shown in solid in FIG. 11) to its clamped configuration (shown in FIG. 12).

1. A catheter assembly, comprising:
a first line having (i) a first proximal end portion defining a first opening, (ii) a first distal end portion defining a second opening, and (iii) a first fluid passage extending between the first opening and the second opening,
a second line having (i) a second proximal end portion defining a third opening, (ii) a second distal end portion defining a fourth opening, and (iii) a second fluid passage extending between the third opening and the fourth opening,
an actuator movable between a first location and a second location; and
a linkage connected between said actuator and said first distal end portion of said first line,
wherein said first distal end portion of said first line is movable in relation to said second line between (i) a first position in which said first distal end portion is positioned in abutment with said second line, and (ii) a second position in which said distal end portion is spaced apart from said second line, and
wherein movement of said actuator from said first location to said second location causes movement of said first distal end portion from said first position to said second position.

2. The catheter assembly of claim 1, wherein:
said actuator includes a clamp positioned around said first line,
said clamp is movable between a clamped configuration and an unclamped configuration, and
movement of said clamp from said clamped configuration to said unclamped configuration causes movement of said distal end portion from said first position to said second position.

3. The catheter assembly of claim 2, wherein:
fluid flow through said first line is prevented when said clamp is in said clamped configuration, and
fluid flow though said first line is enabled when said clamp is in said unclamped configuration.

4. The catheter assembly of claim 1, wherein:
said first distal end portion of said first line possess is D-shaped and defines a first flat wall,
said second distal end portion of said second line is also D-shaped and defines a second flat wall, and
said first flat wall is juxtaposed to said second flat wall when said first distal end portion of said first line is positioned in said first position, and
said first flat wall is spaced apart from said second flat wall when said first distal end portion of said first line is positioned in said second position.

5. The catheter assembly of claim 4, wherein:
said first flat wall has defined therein a first plurality of holes, and
said second flat wall has defined therein a second plurality of holes.

6. The catheter assembly of claim 5, wherein:
said first distal end portion of said first line further defines a first curved wall to which said first flat wall is attached, and
said first curved wall is free of holes.

7. The catheter assembly of claim 6, wherein:
said second distal end portion of said second line further defines a second curved wall to which said second flat wall is attached, and
said second flat wall is free of holes.

8. The catheter assembly of claim 7, wherein:
said first flat wall and said first curved wall collectively define said second opening.

9. The catheter assembly of claim 1, wherein said linkage includes:
a first segment of suture having (i) a first end coupled to said first distal end portion and a second end, (ii) a second end coupled to said second line, and (iii) a central portion interposed between said first end and said second end, and
a second segment of suture having (i) a first end coupled to said actuator and (ii) a second end coupled to said central portion of said first segment of suture.

10. The catheter assembly of claim 9, wherein:
said linkage further includes a ring defining a through-hole,
said second end of said second segment of suture is attached to said ring, and
said central portion of said first segment of suture extends through said through-hole of said ring.

11. The catheter assembly of claim 9, further comprising:
a first guide tube defining a first guide lumen, said first guide tube being secured to said first line,
a second guide tube defining a second guide lumen, said second guide tube being secured to said second line,
wherein said first segment of suture extends through both said first guide lumen and said second guide lumen.

12. The catheter assembly of claim 1, further comprising a tube segment attached to an outer wall of said second line, wherein:
said second opening of said first distal end portion of said first line is located proximal to said tube segment.

13. The catheter assembly of claim 12, wherein:
said tube segment defines a passageway therethrough, and
said passageway of said tube segment is aligned with said first fluid passage of said first line.

14. The catheter assembly of claim 13, wherein:
said tube segment defines a first flat wall,
said outer wall of said second line defines a second flat wall, and
said first flat wall is secured to said second flat wall.

15. A catheter assembly, comprising:
a first fluid line having (i) a first proximal end portion, (ii) a first distal end portion, and (iii) a first fluid passage,
a second fluid line having (i) a second proximal end, (ii) a second distal end portion, and (iii) a second fluid passage,
a control line attached said first distal end portion of said first fluid line; and
a first guide tube defining a first guide lumen, said first guide tube being secured to said first fluid line,
wherein said control line extends through said guide tube,
wherein said first distal end portion of said first fluid line is movable in relation to said second fluid line between (i) a first position in which said first distal end portion is positioned at a first location with respect to said second fluid line, and (ii) a second position in which said distal end portion is positioned at a second position with respect to said second fluid line, and
wherein movement of said control line proximally causes movement of said first distal end portion from said first position towards said second position.

16. The catheter assembly of claim 15, further comprising an actuator configured to move said control line proximally.

17. The catheter assembly of claim 16, wherein:
said actuator includes a clamp positioned around said first fluid line,
said clamp is movable between a clamped configuration and an unclamped configuration, and
movement of said clamp from said clamped configuration to said unclamped configuration causes movement of said control line proximally.

18. The catheter assembly of claim 17, wherein:
fluid flow through said first fluid line is prevented when said clamp is in said clamped configuration, and
fluid flow though said first fluid line is enabled when said clamp is in said unclamped configuration.

19. The catheter assembly of claim 15, wherein:
said first distal end portion of said first fluid line possess is D-shaped and defines a first flat wall,
said second distal end portion of said second fluid line is also D-shaped and defines a second flat wall, and
said first flat wall is juxtaposed to said second flat wall when said first distal end portion of said first fluid line is positioned in said first position, and
said first flat wall is spaced apart from said second flat wall when said first distal end portion of said first fluid line is positioned in said second position.

20. The catheter assembly of claim 19, wherein:
said first flat wall has defined therein a first plurality of holes, and
said second flat wall has defined therein a second plurality of holes.

21. The catheter assembly of claim 20, wherein:
said first distal end portion of said first fluid line further defines a first curved wall to which said first flat wall is attached, and
said first curved wall is free of holes.

22. The catheter assembly of claim 21, wherein:
said second distal end portion of said second fluid line further defines a second curved wall to which said second flat wall is attached.

23. The catheter assembly of claim 22, wherein:
said first flat wall and said first curved wall collectively define a first opening in said first fluid line, and
said second flat wall and said second curved wall collectively define a second opening in said second fluid line.

24. The catheter assembly of claim 15, wherein said control line includes a first segment of suture having a first end coupled to said first distal end portion.

25. The catheter assembly of claim 24, wherein said control line further includes a second end coupled to said second fluid line.

26. The catheter assembly of claim 25, wherein said control line further includes a central portion interposed between said first end and said second end.

27. The catheter assembly of claim 26, further comprising an actuator configured to move said control line proximally, wherein said control line further includes a second segment of suture having (i) a first end coupled to said actuator and (ii) a second end coupled to said central portion of said first segment of suture.

28. The catheter assembly of claim 27, wherein:
said control line further includes a ring defining a through-hole,
said second end of said second segment of suture is attached to said ring, and
said central portion of said first segment of suture extends through said through-hole of said ring.

29. The catheter assembly of claim 15, further comprising:
a second guide tube defining a second guide lumen, said second guide tube being secured to said second fluid line,
wherein said control line extends through both said first guide lumen and said second guide lumen.

30. The catheter assembly of claim 15, further comprising a tube segment attached to an outer wall of said second fluid line, wherein:
said second opening of said first distal end portion of said first fluid line is located proximal to said tube segment.

31. The catheter assembly of claim 30, wherein:
said tube segment defines a passageway therethrough, and
said passageway of said tube segment is aligned with said first fluid passage of said first fluid line.

32. The catheter assembly of claim 30, wherein:
said tube segment defines a first flat wall,
said outer wall of said second fluid line defines a second flat wall, and
said first flat wall is secured to said second flat wall.
